(19) United States
(12) Patent Application Publication
(10) Pub. No.: US 2011/0190502 A1
(43) Pub. Date: Aug. 4, 2011

(54) PROCESS FOR THE PREPARATION OF S-CLOPIDOGREL

(75) Inventors: Shreerang Joshi, Thane (IN); Sachin Bhuta, Thane (IN); Raji Nair, Thane (IN); Sanjukumar Salunke, Thane (IN)

(73) Assignee: SANDOZ AG, Basel (CH)

(21) Appl. No.: 13/121,769

(22) PCT Filed: Oct. 23, 2009
(86) PCT No.: PCT/EP09/63996

§ 371 Date: Mar. 30, 2011

(30) Foreign Application Priority Data
Oct. 24, 2008 (IN) 2292/MUM/2008

(51) Int. Cl. C07D 471/04

(52) U.S. Cl. 546/114

(57) ABSTRACT

A process for the preparation of (S)-Clopidogrel free base or a pharmaceutically acceptable salt thereof by the racemization of the undesired (R)-Clopidogrel in the presence of a suitable base followed by resolution with camphor sulfonate salt and further treatment with an inorganic acid to yield the title compound.

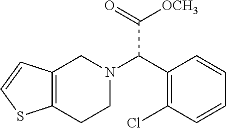

### FIELD OF THE INVENTION

The present invention relates to a process for the preparation of S-Clopidogrel from undesired R-Clopidogrel.

### BACKGROUND OF THE INVENTION

Atherosclerosis is the buildup of plaque in the wall of the arteries leading to a thickening and a reduction in elasticity of the arteries. Atherosclerosis results from injury to the inside layer of the artery. The injury is caused by common activities and diseases such as high cholesterol, high blood pressure, smoking and infection.

Plaques form on the inner walls of the artery at these sites of injury. The plaques are mainly composed of fatty tissue and smooth muscle cells. The formation of plaque often leads to blood clotting due to platelet aggregation at the site of the injury. This clotting may result in a reduction or elimination of blood flow to vital organs, causing heart attacks or other serious conditions. The plaque may also rupture and send a blood clot through the artery, referred to as an embolus, which if deposited in a smaller blood vessel may completely block blood flow.

Antiplatelet activity is desirable in fighting the often fatal results of atherosclerosis. Clopidogrel is an inhibitor of induced platelet aggregation which acts by inhibiting the binding of adenosine diphosphate to its receptor. Clopidogrel is metabolized by the liver into active form. Its antiplatelet activity is extended in that it stops any platelet activity even up to ten days after administration.

The chemical name of Clopidogrel is methyl (+)-(S)-α-(o-chlorophenyl)-6,7-dihydrothieno[3,2-c]pyridine-5(4H)-acetate.

It has the following structure:

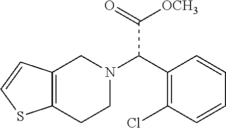

Clopidogrel is disclosed in U.S. Pat. No. 4,529,596 (EP 99802, JP 59027895), U.S. Pat. Nos. 6,258,961, 5,036,156 (EP 420706, JP 3120286), U.S. Pat. No. 6,080,875 (EP 971915, JP 2001513806), U.S. Pat. No. 6,180,793 (EP 981529, JP 2001525829), FR 2769313, and preparation of Clopidogrel.

U.S. Pat. No. 4,529,596 discloses a racemic mixture of Clopidogrel and processes for preparing such mixture.

U.S. Pat. No. 5,036,156 discloses a method for preparing an intermediate in the synthesis of Clopidogrel, 2-chloro-.alpha.-bromophenylacetic acid, and a process for condensing its methyl ester with tetrahydrothienopyridine.

FR 2769313 discloses an intermediate in the synthesis of Clopidogrel, (R)-2-benzenesulfonyloxy-2-(2-chlorophenyl)acetic acid methyl ester, and processes for its preparation. FR 2769313 further discloses converting the ester to Clopidogrel by nucleophilic substitution with tetrahydrothienopyridine.

U.S. Pat. No. 5,036,156 discloses preparation of pyridine derivatives by reacting a benzaldehyde with tribromomethane and potassium hydroxide in water and in the presence of an inert solvent.

Clopidogrel's platelet inhibiting activity makes it an effective drug for reducing the incidence of ischemic strokes, heart attacks or claudication due to vascular diseases such as atherosclerosis. By inhibiting platelet aggregation, Clopidogrel reduces the chance of arterial blockage, thus preventing strokes and heart attacks.

U.S. Pat. No. 5,576,328 describes a method of preventing the occurrence of a secondary ischemic event by administration of Clopidogrel.

Recent studies have shown that Clopidogrel is more effective in blocking platelet aggregation than aspirin and is much gentler on the gastrointestinal tract. Clopidogrel is more effective than aspirin even at much lower dosage. A dosage of 75 mg of base equivalent has been shown to be more effective than a dosage of 325 mg of aspirin. In addition to being more effective, Clopidogrel produces much less gastrointestinal bleeding than aspirin.

Clopidogrel is administered as its bisulfate salt. It is currently being marketed as PLAVIX® tablets, which contain about 98 mg Clopidogrel bisulfate, which is the equivalent of 75 mg Clopidogrel base. PLAVIX® is a white to off-white powder that is practically insoluble in water at neutral pH but highly soluble at acidic pH. It dissolves freely in methanol, somewhat in methylene chloride, and poorly in ethyl ether. The enantiomer (S) Clopidogrel is particularly preferred since it is the pharmaceutically active compound.

U.S. Pat. No. 6,080,875 (EP 971915, JP 2001513806), discloses preparation (S) Clopidogrel by reaction of sodium 2-thienylglycidate with (S) 2-chloro phenyl glycine in the presence of cyanoborohydride.

U.S. Pat. No. 6,180,793 (EP 981,529, JP 2001525819) and related publications WO 98/51681, WO 98/51682 and WO/51689, discloses preparation of (S) enantiomer of Clopidogrel by methods that control the chirality of the intermediates used in the synthesis of clopiodogrel to reduce formation of the (R) enantiomer. U.S. Pat. No. 6,180,793 and the related art disclose processes for synthesizing (S) Clopidogrel by reaction of an activated form of 2-thiophene ethanol with (S)-2-chlorophenyl glycineamide, (S)-2-chlorophenyl-alpha.-amino acetonitrile or (S)-2-chlorophenyl glycine methyl ester. After condensation, the resulting compound is cyclicized, hydrolyzed and esterified.

WO 98/39286 discloses a racemization process for phenyl glycine esters. A mixture of enantiomers of phenyl glycine ester is treated with a carbonyl compound in the presence of a carboxylic acid and a single enantiomer of an N-protected-α-amino acid as resolving agent. The formation of an imino intermediate causes the racemization of the starting product and the precipitation of a single diastereomeric salt. After hydrolysis of the salt, an enantiomer of phenyl glycine ester is obtained.

U.S. Pat. No. 4,847,265 (EP 291459, JP 63203684) discloses methods for separating one enantiomer of Clopidogrel from another by selective crystallization of the camphor sulfonate of the (S) enantiomer. The '265 patent discloses crystallizing the (S) enantiomer from dimethylformamide (“DMF”), ketones, and alcohols, though crystallization with acetone is primarily disclosed. U.S. Pat. No. 5,132,435 (EP 465358, JP 3055819), U.S. Pat. No. 6,215,005 and U.S. Pat. No. 6,258,961, also disclose separating the (S) enantiomer of Clopidogrel by crystallization of the camphor sulfonate from acetone.

U.S. Pat. No. 5,204,469 (EP 466569, JP 4230387) discloses an enantioselective process for synthesis of Clopidogrel through reaction of (+)-2-chloro phenylglycine and an activated form of 2-thiophene ethanol followed by cyclization with formaldehyde.

WO 00/27840 (EP 1129087) discloses using a base to racemize an amide intermediate used in the synthesis of Clopidogrel. The process of WO 00/27840 requires going through an amide intermediate, which is not always a preferred route in preparing Clopidogrel. It is advantageous to prepare Clopidogrel, and then racemize Clopidogrel rather than the intermediate, and to skip the necessary conversion of the amide intermediate to an ester as required in WO 00/27840. WO 02/059128 also generally discloses racemization of an intermediate of Clopidogrel and Clopidogrel with an equimolar amount of a base, though an actual example is not provided regarding racemization of Clopidogrel.

U.S. Pat. No. 6,737,411 discloses a process for racemization of enriched R-Clopidogrel, which is left in the mother liquor, after removal of S-Clopidogrel. The process comprises reacting R-Clopidogrel with a catalytic amount of a base in a solvent to convert a portion of the R-Clopidogrel to S-Clopidogrel. Preferred bases are sodium t-butoxide, potassium t-butoxide, diisopropylamide, sodium hydride, potassium hydride, sodium methoxide and potassium methoxide. Preferably, the solvent is a hydrocarbon.

IN 193363 Patent discloses a process for racemization R-Clopidogrel, using a combination of base in a solvent and takes 15 hours for racemization R-Clopidogrel.

A problem with the preparation of Clopidogrel is the presence of a therapeutically inactive enantiomer, the (R) enantiomer. The presence of the (R) enantiomer results in contamination of the main product, and reduces the yield by being a waste product. There is a need in the art to prepare the (S) enantiomer of Clopidogrel substantially free of the (R) enantiomer in a facile manner suitable on an industrial scale. The method should also ensure that no significant hydrolysis of the methyl ester function present in the Clopidogrel takes place during the process of racemization. The present inventors have found that the addition of a small amount of water in the racemisation reaction leads to the preparation of the racemised product with higher and consistent yields as compared to those obtained under anhydrous conditions.

### OBJECTS OF THE INVENTION

The main object of the present invention is to provide a process for the conversion of Clopidogrel mixture having enriched undesired R(−)-isomer to its racemate without causing significant hydrolysis of the ester group, so that more of the desired (S)-(+)-Clopidogrel can be obtained by resolution.

Another object of the present invention is to provide a process without using hazardous and expensive chemicals.

Yet another object of the present invention is to avoid use of anhydrous solvents and to use common solvents like acetone, toluene, etc.

Yet another object of the present invention is to avoid the use of strong and costly bases like metal hydrides, metal-t-butoxides and metal alkoxides and to use cheap bases like Sodium hydroxide, Potassium hydroxide, etc.

Yet another object of the present invention is to keep the work up process very simple and hence easy to operate for large scale industrial preparation of Clopidogrel.

Yet another object of the present invention is to reduce time cycle as far as possible for large scale industrial preparation of Clopidogrel.

### SUMMARY OF THE INVENTION

According to one aspect of the present invention, a process is provided for preparing (S)-Clopidogrel free base or a pharmaceutically acceptable salt thereof comprising the steps of:
a) Racemizing a mixture containing enriched undesired R-Clopidogrel by the addition of a base and a catalytic amount of water and in the presence of a solvent at 35° C.-55° C. to form a racemic mixture;
b) Resolving the racemic mixture using levorotatory camphor sulfonic acid in the presence of a solvent to precipitate (S)-Clopidogrel camphor sulfonate;
c) Converting (S)-Clopidogrel camphor sulfonate to clopidogrel free base by reacting with an inorganic base;
d) Adding inorganic acid to the free base to precipitate a pharmaceutically acceptable salt of (S) Clopidogrel.

According to another aspect of the present invention, a process is provided for preparing a pharmaceutically acceptable salt of (S) clopidogrel comprising the steps of:
a) Reacting a solution of (R) and (S) clopidogrel with levorotatory camphor sulfonic acid in acetone to precipitate first (S) Clopidogrel camphor sulfonate;
b) Racemizing (R) clopidogrel remaining in acetone by the addition of a base and a catalytic amount of water to obtain a second mixture of (R) and (S) clopidogrel;
c) Reacting a second mixture of (R) and (S) Clopidogrel with levorotatory camphor sulfonic acid to precipitate second (S) Clopidogrel camphor sulfonate;
d) Converting the first and second (S) Clopidogrel camphor sulfonate to a free base;
e) Adding inorganic acid to the free base to precipitate a pharmaceutically acceptable salt of (S) Clopidogrel.

The present invention provides a process for recycling a mixture containing enriched undesired R-Clopidogrel and/or (R)-Clopidogrel by a process of racemization, followed by resolution of the mixture thus obtained by using a conventional process to yield desired S-Clopidogrel.

### DETAILED DESCRIPTION OF THE INVENTION

The present invention provides a process for racemizing a mixture containing enriched undesired R-Clopidogrel comprising, reacting the mixture containing enriched undesired R-Clopidogrel by the addition of a base and a catalytic amount of water and in the presence of solvent to convert a portion of the (R) Clopidogrel to (S) Clopidogrel. Preferred bases are sodium hydroxide and potassium hydroxide. Preferably, the solvent is an organic solvent and it is selected from the group consisting of toluene, methyl ethyl ketone, methyl isobutyl ketone and acetone. Preferably, the racemization is carried out at a temperature of about 35 to 55° C., more preferably at a temperature of about 50 to 55° C. The ratio of the mole equivalent of the substrate and sodium/potassium hydroxide is preferably in the range of 0.1 to 0.5, more preferably in the range of 0.15 to 0.25.

The addition of a catalytic amount of water in the racemisation reaction leads to the preparation of the racemised product with higher and consistent yields.

In another aspect, the present invention provides a process for preparing a pharmaceutically acceptable salt of (S) Clopidogrel comprising the steps of reacting a solution of Clopidogrel (R) and (S) in acetone with levorotatory camphor sulfonic acid, thereby forming a first Clopidogrel (S) camphor sulfonate as a precipitate, removing the acetone and excess camphor sulfonic acid from the filtrate/mother liquor, racemizing the (R) Clopidogrel remaining in acetone by the addition of a base (sodium or potassium hydroxide) and catalytic amount of water to form a mixture of (R) and (S) Clopidogrel, adding levorotatory camphor sulfonic acid to precipitate Clopidogrel (S) camphor sulfonate as a second precipitate, converting the first and second Clopidogrel (S) camphor sulfonate to a free base and further converting it to a pharmaceutically acceptable salt of (S) Clopidogrel.

In another aspect, the present invention provides a process for preparing (S) Clopidogrel bisulfate comprising the steps of reacting a solution of Clopidogrel (R) and (S) in acetone with levorotatory camphor sulfonic acid, thereby forming a first Clopidogrel (S) camphor sulfonate as a precipitate, removing the acetone and excess camphor sulfonic acid from the filtrate/mother liquor, racemizing the (R) Clopidogrel remaining in acetone by the addition of a base (sodium or potassium hydroxide) and a catalytic amount of water to form a mixture of (R) and (S) Clopidogrel, adding levorotatory camphor sulfonic acid to precipitate Clopidogrel (S) camphor sulfonate as a second precipitate, converting the first and second Clopidogrel (S) camphor sulfonate to a free base, adding sulfuric acid to a free base to precipitate (S) Clopidogrel bisulfate.

In another aspect, the present invention provides a process for preparing (S) Clopidogrel hydrochloride comprising the steps of reacting a solution of Clopidogrel (R) and (S) in acetone with levorotatory camphor sulfonic acid, thereby forming a first Clopidogrel (S) camphor sulfonate as a precipitate, removing the acetone and excess camphor sulfonic acid from the filtrate/mother liquor, racemizing the (R) Clopidogrel remaining in acetone by the addition of a base (sodium or potassium hydroxide) and a catalytic amount of water to form a mixture of (R) and (S) Clopidogrel, adding levorotatory camphor sulfonic acid to precipitate Clopidogrel (S) camphor sulfonate as a second precipitate, converting the first and second Clopidogrel (S) camphor sulfonate to a free base, adding hydrochloric acid to a free base to precipitate (S) Clopidogrel hydrochloride.

The present invention thus provides facile processes for separation of the (S) enantiomer of Clopidogrel from a racemic mixture of the enantiomers, and recycling the (R) enantiomer after the separation step to produce more of the (S) enantiomer. The Clopidogrel used in the present invention can be synthesized according to the disclosed methods of the present invention or any method known in the art.

The invention is further described by reference to the following examples which set forth in detail the preparation of compounds of the present invention. It will be apparent to those skilled in the art that many modifications, both to materials and methods, may be practiced without departing from the purpose and interest of this invention. The examples that follow illustrate the present invention and are not intended to limit the scope of the invention as described hereinabove.

### EXAMPLES

### Example 1

### Racemization of Clopidogrel

To 40 gm of Clopidogrel free base containing undesired isomer (obtained after evaporation of filtrate from resolution and conversion into free base) was added 200 ml. acetone and 1.7 gms KOH flakes (0.25 eq. with respect to input Clopidogrel base). The reaction mixture was heated at 50° C. to 55° C. for 2.5 hours till SOR was ±1 to 0. The solvent was distilled under reduced pressure and the reaction mixture was cooled 25° C. to 30° C. To the resultant oil was charged 60 ml of toluene and 40 ml of water and stirred for about 10 to 15 minutes. The toluene layer was separated and washed with 40 ml of water. Toluene was distilled under reduced pressure at 50° C. to 55° C. to obtain 34.86 gms of racemised base

### Example 2

### Racemization of Clopidogrel

To 40 gm of Clopidogrel free base containing undesired isomer (obtained after evaporation of filtrate from resolution and conversion into free base) were added 200 ml. acetone and 1.047 gms NaOH flakes (0.25 eq. with respect to input Clopidogrel base) and 0.26 gms water. The reaction mixture was heated at 50° C. to 55° C. for 2.5 hours till SOR was ±1 to 0. The solvent was distilled under reduced pressure and the reaction mixture was cooled 25° C. to 30° C. To the resultant oil was charged 80 ml of toluene and 120 ml of water and stirred for about 10 to 15 minutes. The toluene layer was separated and washed with 40 ml of water. Toluene was distilled under reduced pressure at 50° C. to 55° C. to obtain 32.35 gms of racemised base.

### Example 3

### Racemization of Clopidogrel

Charge 40 gm of Clopidogrel free base containing undesired isomer (obtained after evaporation of filtrate from resolution and conversion into free base) followed by 200 ml acetone. (5 vol. with respect to input clopidogrel base) Charge NaOH flakes 1.24 gms (0.25 eq. with respect to input clopidogrel base) Heat the resultant reaction mixture at reflux (54° C. to 55° C.) for 3.0 hrs. Check the SOR of the sample. If SOR is ±1 to 0 then workup the reaction mixture. Work up: Distill the solvent under reduced pressure at 45° C. to 50° C. Cool the reaction mixture to 25° C. to 30° C. Charge 80 ml toluene (2 vol) and 120 ml water (3 vol) under stirring. Stir for about 10 to 15 minutes and separate the toluene layer. Wash the Toluene layer with 80 ml (2 vol.) water. Separate the toluene layer and distill under reduced pressure at 50° C. to 55° C. to obtain 29.5 gms of racemised base.

### Example 4

### Resolution by Camphorsulfonic Salt Formation

To the 707 gms of concentrated mass obtained as in examples 1, or 2 was charged 707 ml acetone at 25° C.-30° C. To the resultant clear solution, 270 gms of camphor sulfonic acid was charged and the reaction mixture was then heated to 40° C.-45° C. Seed crystals of CSA salt of Clopidogrel were charged and then the reaction mixture was heated at 50° C. to 55° C. for 3 hrs. The reaction mixture was gradually cooled to 0° C. to 5° C. and maintained for 3 hrs. The crystals were filtered and the wet cake was again taken in 2.35 lts. acetone and heated at 55° C.±2° C. for 2 hrs. The slurry was cooled to 25° C.-30° C., filtered and dried to afford 353 gms of the product.

### Example 5

### Conversion of CSA Salt of Clopidogrel to Clopidogrel Bisulphate

To 50 g Clopidogrel free base liberated from CSA salt of Clopidogrel obtained as in example 3 was charged 150 ml acetone and stirred at 25° C.-30° C. till a homogenous solution was obtained. A solution of 150 ml acetone and 15 g conc.H₂SO₄ was cooled to 5° C. to 10° C. and then added into the above solution of Clopidogrel base in acetone at 30° C. The reaction mixture was stirred for 30 minutes and then distilled to remove the acetone under reduced pressure till 5-10 ml acetone remained along with the Clopidogrel bisulphate residue. To the residue thus obtained was added slurry of 50 ml n-butyl acetate and 0.5 g seed followed by 200 ml n-butyl acetate. The reaction mixture was stirred 25° C. to 30° C. for about 20 hrs. The crystals thus obtained when filtered and dried under vacuum at 25° C. to 30° C. for 24 hrs afforded 59.6 g of Clopidogrel bisulphate form I.

### Example 6

### Conversion of CSA Salt of Clopidogrel to Clopidogrel Hydrochloride

To 1.0 kg CSA salt of Clopidogrel obtained as in example 3 was charged 2.0 lts. ethyl acetate at 25° C.-30° C. and stirred for about 5 minutes. To the resultant solution was charged a 10% solution of 523 gm sodium bicarbonate in 5000 ml water and stirred for 60 min. The ethyl acetate layer was separated and the aqueous layer was extracted with 1.0 lts. ethyl acetate. The combined organic layers were washed with saturated brine solution (500 gm sodium chloride dissolved in 1200 ml. water). The combined organic layers were stirred with 250 to 300 gms molecular sieves for about 30 min. at 25° C. to 30° C. and then filtered. A solution of 62.3 gm HCl in ethyl acetate was added in 1 hr. to the filtrate containing Clopidogrel base and then stirred at 25° C. to 30° C. for about 3 hrs. The crystals were filtered and dried to afford 550 gms of the product.

1) A process for preparing (S) clopidogrel free base or a pharmaceutically acceptable salt thereof from a mixture containing enriched undesired R-Clopidogrel comprising the steps of:
a) Racemizing a mixture containing enriched undesired R-Clopidogrel by the addition of a base and a catalytic amount of water in the presence of a solvent and at 35° C.-55° C. to form a racemic mixture;
b) Resolving the racemic mixture using levorotatory camphor sulfonic acid in the presence of a solvent to precipitate (S) clopidogrel camphor sulfonate;
c) Converting (S) clopidogrel camphor sulfonate to clopidogrel free base by reacting with an inorganic base;
d) Adding inorganic acid to the free base to precipitate a pharmaceutically acceptable salt of (S) Clopidogrel.

2) The process according to claim 1 wherein the organic solvent is selected from the group consisting of toluene, methyl ethyl ketone, methyl isobutyl ketone and acetone.

3) The process according to claim 1 wherein the base for racemization is selected from sodium hydroxide or potassium hydroxide.

4) The process according to claim 1 wherein the base for racemization is in the range of 0.1 to 0.5.

5) The process according to claim 1 wherein the inorganic acid is selected from sulfuric acid or hydrochloric acid.

6) The process according to claim 1 wherein the inorganic base is sodium bicarbonate.

7) The process according to claim 1 wherein the temperature for carrying out the racemization reaction is 50° C.-55° C.

8) A process for preparing a pharmaceutically acceptable salt of (S) clopidogrel comprising the steps of:
a) Reacting a solution of (R) and (S) clopidogrel with levorotatory camphor sulfonic acid in acetone to precipitate first (S) Clopidogrel camphor sulfonate;
b) Racemizing (R) clopidogrel remaining in acetone by the addition of a base and a catalytic amount of water to obtain a second mixture of (R) and (S) clopidogrel;
c) Reacting a second mixture of (R) and (S) Clopidogrel with levorotatory camphor sulfonic acid to precipitate second (S) Clopidogrel camphor sulfonate;
d) Converting the first and second (S) Clopidogrel camphor sulfonate to a free base;
e) Adding inorganic acid to the free base to precipitate a pharmaceutically acceptable salt of (S) Clopidogrel.

9) The process according to claim 8 wherein the inorganic acid is selected from sulfuric acid or hydrochloric acid.
